(19) United States
(12) Patent Application Publication
(10) Pub. No.: US 2020/0069152 A1
(43) Pub. Date: Mar. 5, 2020

(54) Wireless Endoscope

(71) Applicant: Olympus Corporation, Tokyo (JP)

(72) Inventor: Makoto Kasumi, Tokyo (JP)

(73) Assignee: Olympus Corporation, Tokyo (JP)

(21) Appl. No.: 16/677,472

(22) Filed: Nov. 7, 2019

Related U.S. Application Data
(63) Continuation of application No. PCT/JP2018/014810, filed on Apr. 6, 2018.

(30) Foreign Application Priority Data
May 12, 2017 (JP) 2017-095780

(51) Int. Cl. A61B 1/00; A61B 1/06

(52) U.S. Cl. CPC A61B 1/00096; A61B 1/0684

(57) ABSTRACT

The disclosed technology is directed to an endoscope comprises an endoscope body defined by an insertion portion and an operating portion both of which are a hermetically sealed structure with a predetermined gas pressure maintained in the endoscope body. The operation portion includes a ventilation inlet attached thereto to deliver a gas into the endoscope body and to hold the gas at the predetermined gas pressure in the endoscope body. A measuring unit is configured to be mounted on a board disposed in the operating portion for measuring a gas pressure in the endoscope body. A controller is configured to acquire a value measured by the measuring unit and to determine whether or not there is a gas leakage from the endoscope body based on the acquired measured value.

### CROSS-REFERENCE TO RELATED APPLICATIONS

This application is a continuation application of PCT Application No. PCT/JP2018/014810 filed on Apr. 6, 2018, which in turn claim priority to the Japanese Patent Application No. 2017-95780 filed on May 12, 2017 in Japan which is hereby incorporated by reference in its entirety.

### TECHNICAL FIELD

The disclosed technology relates to a wireless endoscope that can be powered by a battery.

### DESCRIPTION OF THE RELATED ART

Heretofore, as the semiconductor technology has advanced, various devices including cell phones, smart phones, tablet personal computers (PCs), etc. have been so reduced in size and electric power consumption that they have been configured as portable. Many portable devices carry a battery that can be charged to make them available for continuous use.

In the medical field, efforts have been made to make devices smaller in size. For example, endoscopes consume a relatively large amount of electric power, and there have been developed wireless endoscopes with a chargeable battery. Wireless endoscopes in the medical field are used for observations of organs in body cavities, medical treatments using treatment tools, and surgical operations under endoscopic observation.

Since endoscopes are medical devices that are reused, they need to be washed and sterilized after use. In a case in which an endoscope has a pinhole or a loose joint when it is washed and sterilized, liquids such as water, a sterilizing liquid, etc. tend to enter the endoscope, possibly causing electric components including an optical fiber, a charge-coupled device (CCD), etc. to malfunction. In order to prevent liquids such as water, a sterilizing liquid, etc. from entering an endoscope, it is necessary before the endoscope is washed and sterilized to conduct a leakage test on the endoscope to confirm whether or not there is an air leakage from the endoscope. Japanese Patent Application-JP 2005-91042 A, for example, discloses a leakage tester for carrying out such a leakage test.

According to the conventional leakage test, a ventilation inlet of the endoscope and the leakage tester are connected to one another by a tube. After air is introduced under pressure into the housing of the endoscope through the tube via the ventilation inlet, the pressure in the housing is measured to determine whether or not there is an air leakage from the housing. The measurement of the pressure in the housing is followed by a process of releasing the air introduced under pressure into the housing.

The confirmation process is so tedious and time-consuming for the user that the user cannot immediately begin a subsequent washing and sterilizing process. Since wireless endoscopes are required to be easy to handle, the leakage test also needs to be done wirelessly.

Accordingly, there is a need for a wireless endoscope that allows a leakage test to be simply conducted thereon without the need to connect a leakage tester thereto for the leakage test.

### BRIEF SUMMARY OF EMBODIMENTS

One aspect of the disclosed technology is directed to an endoscope comprises an endoscope body defined by an insertion portion and an operating portion both of which are a hermetically sealed structure with a predetermined gas pressure maintained in the endoscope body. The operation portion includes a ventilation inlet attached thereto to deliver a gas into the endoscope body and to hold the gas at the predetermined gas pressure in the endoscope body. A measuring unit is configured to be mounted on a board disposed in the operating portion for measuring a gas pressure in the endoscope body. A controller is configured to acquire a value measured by the measuring unit and to determine whether or not there is a gas leakage from the endoscope body based on the acquired measured value.

Another aspect of the disclosed technology is directed to an endoscope comprises an endoscope body defined by an insertion portion and an operating portion both of which are a hermetically sealed structure with a predetermined gas pressure maintained in the endoscope body. The insertion portion includes a light source applying illuminating light into a body cavity. The insertion portion includes an image capturing unit that captures an image of an inside of the body cavity formed by a return light from the light source. A communication unit transmitting the image captured by the image capturing unit and a power supply generates electric power required for endoscopic observations. The operation portion includes a ventilation inlet attached thereto to deliver a gas into the endoscope body and to hold the gas at the predetermined gas pressure in the endoscope body. A measuring unit is configured to be mounted on a board disposed in the operating portion for measuring a gas pressure in the endoscope body. A controller configured to acquire a value measured by the measuring unit and to determine whether or not there is a gas leakage from the endoscope body based on the acquired measured value. An indicator unit indicating a determined result from the controller.

A further aspect of the disclosed technology is directed to a method of indicating a result of a leakage test on an endoscope. The method comprises conducting the leakage test during a standby mode; determining whether or not a gas pressure in an endoscope body is of a predetermined value by using a gas pressure sensor; controlling an OK light emitting diode indicator to be turned on if a gas pressure value from the gas pressure sensor is equal to or larger than the predetermined value, and controlling an NG light emitting diode indicator to be turned on if the gas pressure value from the gas pressure sensor is not equal to or larger than the predetermined value; and indicating a determined result that represents whether the leakage test is OK or NG based on the light emitting diode indicators as first and second light emitting diode indicators.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrative of the entire makeup of an endoscopic system installed in an operating room.

FIG. 2 is a view illustrative of the general structure of a wireless endoscope according to an embodiment of the disclosed technology.

FIG. 3 is a block diagram of the wireless endoscope according to the embodiment of the disclosed technology.

FIG. 4 is a flowchart illustrative of an example of operation in a leakage test.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

The disclosed technology has as objects thereof the provision of a wireless endoscope and a wireless endoscope system. The wireless endoscope has a normal operation mode and a plurality of standby modes provided beforehand, and can achieve both a quick return to a normal operation mode and low power consumption by switching the standby modes according to the use environment of the endoscope.

According to an aspect of the disclosed technology, there is provided a wireless endoscope including an endoscope body including an insertion portion and an operating portion and having a hermetically sealed structure, with a predetermined gas pressure maintained in the endoscope body, a ventilation inlet delivering a gas into the endoscope body and keeping the predetermined gas pressure in the endoscope body, a measuring unit measuring a gas pressure in the endoscope body, the measuring unit being mounted on a board disposed in the operating portion, a controller acquiring a measured value measured by the measuring unit and determining whether or not the acquired measured value is equal to or larger than a predetermined value, and an indicating unit indicating a determined result of the controller.

An embodiment of the disclosed technology will hereinafter be described below with reference to the drawings.

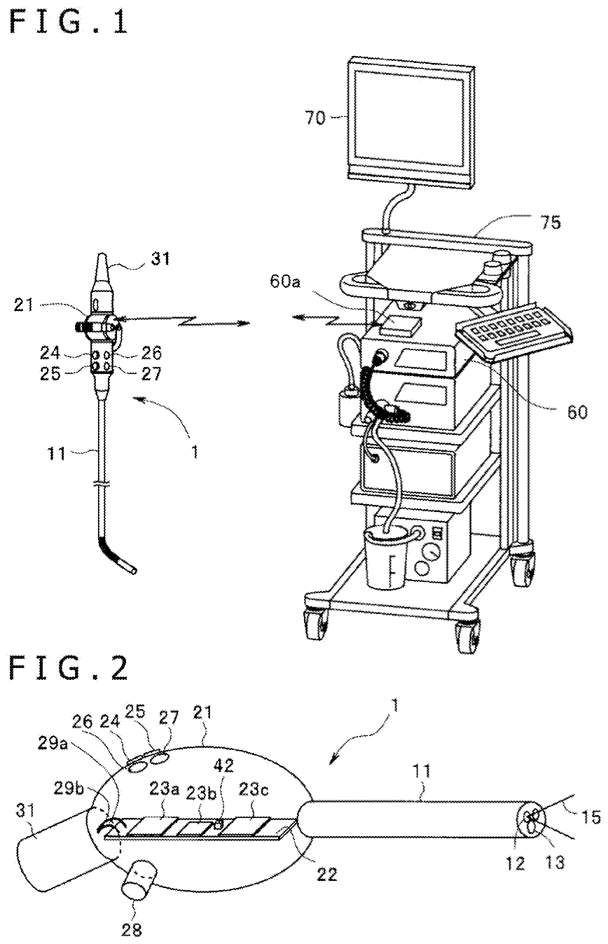

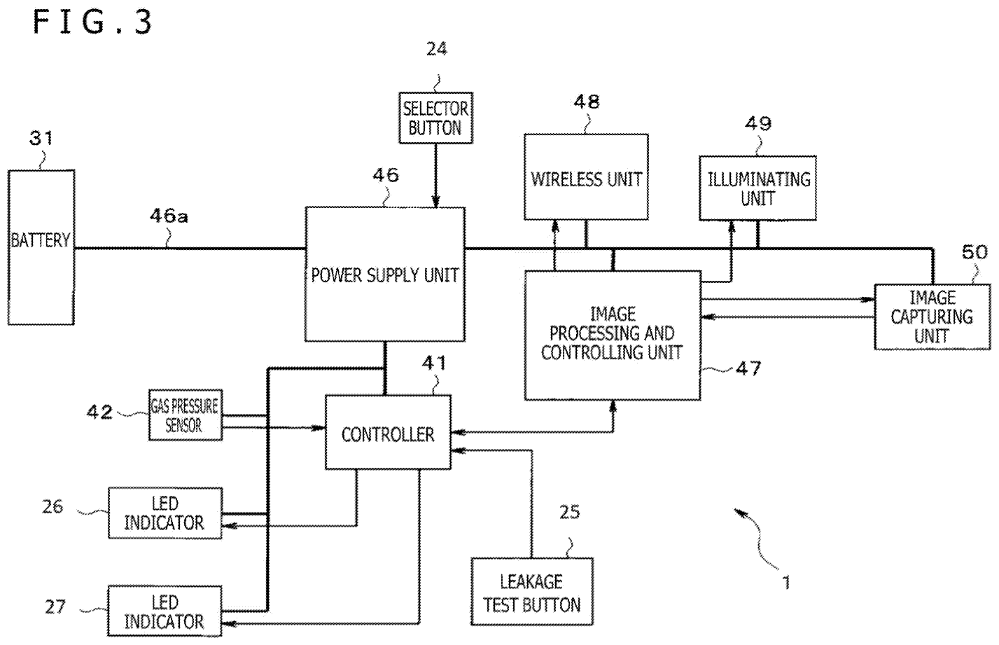

FIG. 1 is a view illustrative of the entire makeup of an endoscopic system installed in an operating room. FIG. 2 is a view illustrative of the general structure of a wireless endoscope according to an embodiment of the disclosed technology. FIG. 3 is a block diagram of the wireless endoscope according to the embodiment of the disclosed technology.

First, the wireless endoscope according to the present embodiment and the endoscopic system that incorporates the wireless endoscope will generally be described below with reference to FIGS. 1 through 3.

As illustrated in FIG. 1, the endoscopic system includes a wireless endoscope 1, a processor 60, and a monitor 70. As illustrated in FIG. 1, various medical devices and the monitor 70 are disposed on a cart 75 in an operating room. The processor 60 that is combined with a wireless device 60a as a separate unit is disposed on the cart 75. The wireless device 60a is connected to the processor 60 by a connector, not illustrated. The medical devices disposed on the cart 75 include devices such as an electric surgical knife device, a pneumoperitoneum device, a video recorder, and so on, and also a gas container filled with carbon dioxide, etc.

The wireless endoscope 1 can capture images for ordinary endoscopic observations when it is powered by a battery 31. The wireless endoscope 1 is connected to the processor 60 through a wireless link.

The wireless endoscope 1 has an insertion portion 11 on its distal end side and an operating portion 21 on its proximal end side. The insertion portion 11 and the operating portion 21 jointly make up an endoscope body having a hermetically sealed structure. The endoscope body including the insertion portion 11 and the operating portion 21 is charged with a gas when the wireless endoscope 1 is manufactured or shipped out of the factory, and maintains a predetermined gas pressure therein.

As illustrated in FIG. 2, the operating portion 21 has a ventilation inlet 28 for delivering a gas into the endoscope body and keeping a predetermined gas pressure in the endoscope body. The gas that fills the endoscope body is delivered only once through the ventilation inlet 28 on the operating portion 21 when the wireless endoscope 1 is manufactured or shipped out of the factory. The ventilation inlet 28 has a check valve structure that allows the gas to be delivered therethrough into the endoscope body, but prevents the delivered gas from leaking out of the endoscope body. Therefore, the predetermined gas pressure established in the endoscope body including the insertion portion 11 and the operating portion 21 is maintained therein by the ventilation inlet 28.

An image capturing unit 50 (see FIG. 3) having an image capturing device such as a CCD, a complementary metal oxide semiconductor (CMOS) sensor, or the like is disposed in a distal end portion of the insertion potion 11. The insertion portion 11 also includes an illuminating unit 49 (see FIG. 3) for emitting illuminating light that illuminate a subject. The light that is emitted from the illuminating unit 49 is applied through a lens 12 on the distal end of the insertion portion 11 as illuminating light 15 to the subject.

Returning light from the subject is applied through a lens 13 on the distal end of the insertion portion 11 to the image capturing unit 50 where the returning light is focused as an optical image of the subject onto an image capturing surface. The image capturing unit 50 obtains a captured image based on the optical image of the subject by way of photoelectric conversion. The image capturing unit 50 transmits the captured image through a signal line, not illustrated, in the insertion portion 11 to a board 22 in the operating portion 21. The board 22 in the operating portion 21 supports various integrated circuits (ICs) 23a through 23c thereon. These ICs 23a through 23c provide various circuits illustrated in FIG. 3.

A gas pressure sensor 42 as a measuring unit for measuring the gas pressure in the endoscope body is mounted on the board 22 in the operating portion 21. The gas pressure sensor 42 is disposed in a free space area in the operating portion 21 out of interference with mechanisms, wires, and boards laid in the operating portion 21.

The image capturing unit 50 and the illuminating unit 49 are illustrated as being disposed in the distal end portion of the insertion portion 11. However, the image capturing unit may be disposed like a camera head in the operating portion 21. The illuminating unit may include a light source disposed in the operating portion 21 or the like and a light guide or the like for guiding illuminating light from the light source to the distal end of the insertion portion 11.

The operating portion 21 includes a battery connector, not illustrated, on which the battery 31 is detachably mounted. Power supply wires 29a and 29b are connected to the battery connector. The power supply wires 29a and 29b supply electric power from the battery 31 to a power supply unit 46 mounted on the board 22. Though the battery 31 is illustrated as being detachably mounted on the operating portion 21 according to the present embodiment, the battery 31 may be housed in the operating portion 21.

The operating portion 21 has a selector button 24 for switching between a normal observation mode and a standby mode, to be described later, a leakage test button 25 for carrying out a leakage test in the standby mode, and light-emitting diode (LED) indicators 26 and 27 for indicating the result of the leakage test to the user. For example, the LED indicator 26 is an OK LED indicator that is turned on when the result of the leakage test is OK, and the LED indicator 27 is an NG LED indicator that is turned on when the result of the leakage test is NG.

As illustrated in FIG. 3, the wireless endoscope 1 includes a controller 41. The controller 41 includes a processor such as a central processing unit (CPU) or the like, not illustrated, and controls various parts of the wireless endoscope 1 according to programs stored in a memory.

In FIG. 3, bold lines represent electric power supply lines. The power supply unit 46 is supplied with electric power from the battery 31 through an electric power supply line 46a, which is indicated as the power supply wires 29a and 29b in FIG. 2, and generates electric power required for endoscopic observations or leakage tests. Specifically, the power supply unit 46 switches between destinations to which electric power is to be supplied according to a selection signal from the selector button 24. The selector button 24 is a button for switching between the normal observation mode and the standby mode. The user presses the selector button 24 to switch between the normal observation mode and the standby mode.

When the selector button 24 is pressed to select the normal observation button, the power supply unit 46 generates electric power to be supplied to an image processing and controlling unit 47, a wireless unit 48, the illuminating unit 49, and the image capturing unit 50 of the wireless endoscope 1.

When supplied with the electric power from the power supply unit 46, the illuminating unit 49 is controlled by the image processing and controlling unit 47 to emit light for illuminating the subject. When supplied with the electric power from the power supply unit 46, the image capturing unit 50 is controlled by the image processing and controlling unit 47 to capture an image of the subject and output the captured image to the image processing and controlling unit 47.

When supplied with the electric power from the power supply unit 46, the image processing and controlling unit 47 performs a predetermined image processing process on the captured image from the image capturing unit 50 and then outputs the processed captured image to the wireless unit 48. For example, the image processing and controlling unit 47 can carry out an image compressing process as the predetermined image processing process. When supplied with the electric power from the power supply unit 46, the wireless unit 48 as a communication unit is energized by the image processing and controlling unit 47 to wirelessly send the captured image through the wireless device 60a to the processor 60, etc.

On the other hand, when the selector button 24 is pressed to select the standby mode, the power supply unit 46 generates electric power to be supplied to the controller 41, the gas pressure sensor 42, and the LED indicators 26 and 27.

When supplied with the electric power from the power supply unit 46, the gas pressure sensor 42 detects a gas pressure value in the endoscope body including the insertion portion 11 and the operating portion 21, and outputs the detected gas pressure value to the controller 41.

When the user presses the leakage test button 25 during the standby mode, it applies a control signal for starting a leakage test to the controller 41. Specifically, the leakage test button 25 is an inspection button for inspecting whether or not a predetermined gas pressure is kept in the endoscope body. When supplied with the control signal for starting the leakage test, the controller 41, upon being supplied with the electric power from the power supply unit 46, acquires the gas pressure value measured by the gas pressure sensor 42, i.e., the measured gas pressure value, and determines whether or not the acquired gas pressure value is equal to or larger than a predetermined value.

If the gas pressure value from the gas pressure sensor 42 is equal to or larger than the predetermined value, the controller 41 controls the OK LED indicator 26 to be turned on. If the gas pressure value from the gas pressure sensor 42 is not equal to or larger than the predetermined value, the controller 41 controls the NG LED indicator 27 to be turned on. The LED indicators 26 and 27, or first and second LED indicators, function as an indicating unit for indicating a determined result from the controller 41 that indicates whether the leakage test is OK or NG.

The controller 41 is illustrated as indicating the result of the leakage test to the user by turning on the LED indicator 26 or the LED indicator 27 in the standby mode. However, the disclosed technology is not limited to such details. The controller 41 may send the result of the leakage test via a wireless link to an external device such as a washing and sterilizing device or the like, so that the result of the leakage test may be displayed on a monitor of the washing and sterilizing device or the like. In this case, the power supply unit 46 supplies electric power to the controller 41, the gas pressure sensor 42, the LED indicator 26, the LED indicator 27, the image processing and controlling unit 47, and the wireless unit 48 in the standby mode. Then, the controller 41 outputs the result of the leakage test through the image processing and controlling unit 47 to the wireless unit 48, which sends the result of the leakage test to the external device such as the washing and sterilizing device or the like. The result of the leakage test is then displayed on the monitor of the washing and sterilizing device or the like, so that the result of the leakage test will be indicated to the user.

According to the present embodiment, the operating portion 21 includes the OK LED indicator 26 and the NG LED indicator 27. However, the disclosed technology is not limited to such details. The operating portion 21 may include only one LED indicator. In this case, the controller 41 indicates the result of the leakage test to the user by controlling the LED indicator to be turned on if the result of the leakage test is OK, and controlling the LED indicator to blink if the result of the leakage test is NG.

As described hereinbefore, when the wireless endoscope 1 according to the present embodiment is manufactured or the like, the predetermined gas pressure, which is a high pressure, is kept in the endoscope body that includes the insertion portion 11 and the operating portion 21. Since the endoscope body has the hermetically sealed structure, the predetermined gas pressure is maintained in the endoscope body in a case in which the wireless endoscope 1 is free of a pinhole or a loose joint, and the gas pressure in the endoscope body drops in a case in which the wireless endoscope 1 has a pinhole or a loose joint.

When the leakage test button 25 is pressed, the controller 41 determines whether or not the wireless endoscope 1 is suffering from a pinhole or a loose joint by determining whether or not the gas pressure value from the gas pressure sensor 42 is equal to or higher than the predetermined value. As the OK LED indicator 26 or the NG LED indicator 27 is turned on when the user presses the leakage test button 25 only once, the user can easily confirm the result of the leakage test.

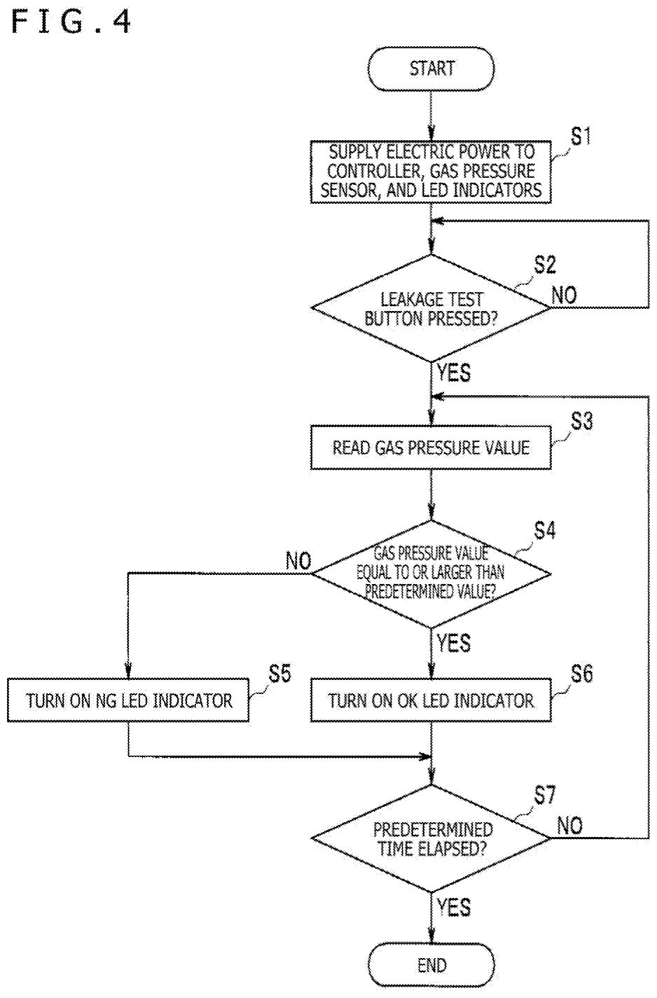

Next, operation of the wireless endoscope 1 thus arranged in a leakage test will be described below. FIG. 4 is a flowchart illustrative of an example of operation in a leakage test. The flowchart of FIG. 4 is executed when the selector button 24 is pressed to bring the wireless endoscope 1 into the standby mode.

When the user presses the selector button 24 to bring the wireless endoscope 1 into the standby mode, the power supply unit 46 supplies electric power to the controller 41, the gas pressure sensor 42, and the LED indicators 26 and 27 in Step S1.

Then, the controller 41 determines whether or not the leakage test button 25 is pressed in Step S2. If the controller 41 determines that the leakage test button 25 is not pressed, then control goes back to Step S2, repeating the same processing. If the controller 41 determines that the leakage test button 25 is pressed, then control goes to Step S3. In Step S3, the controller 41 reads the gas pressure value or measured value from the gas pressure sensor 42, after which control goes to Step S4.

In Step S4, the controller 41 determines whether or not the gas pressure value from the gas pressure sensor 42 is equal to or larger than a predetermined value. If the controller 41 determines that the gas pressure value from the gas pressure sensor 42 is not equal to or larger than the predetermined value, then control goes to Step S5. In Step S5, the controller 41 turns on the NG LED indicator 27, after which control goes to Step S7. On the other hand, if the controller 41 determines that the gas pressure value from the gas pressure sensor 42 is equal to or larger than the predetermined value, then control goes to Step S6. In Step S6, the controller 41 turns on the OK LED indicator 26, after which control goes to Step S7.

In Step S7, the controller 41 determines whether or not a predetermined time has elapsed. If the controller 41 determines that the predetermined time has not elapsed, then control returns to step S3, repeating the same processing. On the other hand, if the controller 41 determines that the predetermined time has elapsed, then the processing sequence is ended. The predetermined time represents a few seconds, for example, so that the OK LED indicator 26 or the NG LED indicator 27 is turned on during a few seconds after the user has pressed the leakage test button 25.

According to the processing sequence described hereinbefore, when the user presses the leakage test button 25, the controller 41 reads the gas pressure value from the gas pressure sensor 42, determines whether or not there is a gas leakage from the read gas pressure value, and controls the OK LED indicator 26 or the NG LED indicator 27 to be turned based on the determined result.

As a result, since the OK LED indicator 26 or the NG LED indicator 27 is turned on when the user presses the leakage test button 25 only once, the user can easily recognize the result of the leakage test. The user does not need to connect a leakage tester for carrying out a leakage test to the wireless endoscope 1, but can easily determine the result of the leakage test simply by pressing the leakage test button 25 only once.

Accordingly, the wireless endoscope 1 according to the present embodiment allows a leakage test to be conducted simply without the need to connect a leakage tester for carrying out a leakage test to the wireless endoscope 1.

In the present embodiment, a leakage test on the wireless endoscope 1 with the battery 31 mounted thereon or housed therein has been described hereinbefore. The disclosed technology is also applicable to a leakage test on an endoscope that is connected to the processor 60 through wires, for example. Generally, an endoscope that is connected to the processor 60 through wires, referred herein as a wired endoscope, does not have a battery that would otherwise be mounted thereon or housed therein, and is supplied with electric power from the processor 60.

Therefore, once such a wired endoscope is disconnected from the processor 60, electric power is not supplied and no leakage test can be conducted on the disconnected endoscope. In this case, after a medical examination using the wired endoscope is finished, the endoscope remains connected to the processor 60 while a leakage test is conducted thereon. Since the wired endoscope is supplied with electric power from the processor 60, the leakage test can be carried out in the same manner as with the wireless endoscope 1 according to the embodiment described hereinbefore.

The disclosed technology is not limited to the embodiments described hereinbefore, but various changes and modifications may be made without departing from the scope of the invention.

In sum, one aspect of the disclosed technology is directed to an endoscope comprises an endoscope body defined by an insertion portion and an operating portion both of which are a hermetically sealed structure with a predetermined gas pressure maintained in the endoscope body. The operation portion includes a ventilation inlet attached thereto to deliver a gas into the endoscope body and to hold the gas at the predetermined gas pressure in the endoscope body. A measuring unit is configured to be mounted on a board disposed in the operating portion for measuring a gas pressure in the endoscope body. A controller is configured to acquire a value measured by the measuring unit and to determine whether or not there is a gas leakage from the endoscope body based on the acquired measured value.

The endoscope further comprises an indicator unit indicating a determined result from the controller. The indicator unit is defined by respective first and second LED indicators. The measuring unit is defined by a gas pressure sensor.

Another aspect of the disclosed technology is directed to an endoscope comprises an endoscope body defined by an insertion portion and an operating portion both of which are a hermetically sealed structure with a predetermined gas pressure maintained in the endoscope body. The insertion portion includes a light source applying illuminating light into a body cavity. The insertion portion includes an image capturing unit that captures an image of an inside of the body cavity formed by a return light from the light source. A communication unit transmitting the image captured by the image capturing unit and a power supply generates electric power required for endoscopic observations. The operation portion includes a ventilation inlet attached thereto to deliver a gas into the endoscope body and to hold the gas at the predetermined gas pressure in the endoscope body. A measuring unit is configured to be mounted on a board disposed in the operating portion for measuring a gas pressure in the endoscope body. A controller configured to acquire a value measured by the measuring unit and to determine whether or not there is a gas leakage from the endoscope body based on the acquired measured value. An indicator unit indicating a determined result from the controller.

The indicator unit includes a first light emitting diode indicator and a second light emitting diode indicator and the controller controls the first light emitting diode indicator or the second light emitting diode indicator to be turned on depending on the determined result. The endoscope further comprises an inspection button for inspecting whether or not the predetermined gas pressure is maintained in the endoscope body and the controller acquires the value measured by the measuring unit when the inspection button is pressed. The endoscope further comprises a wireless unit in which the controller sends the determined result to an external device through the wireless unit via a wireless link. The measuring unit includes a gas pressure sensor and the gas pressure sensor is disposed in a free space area in the operating portion out of interference with mechanisms, wires, and boards laid in the operating portion.

A further aspect of the disclosed technology is directed to a method of indicating a result of a leakage test on an endoscope. The method comprises conducting the leakage test during a standby mode; determining whether or not a gas pressure in an endoscope body is of a predetermined value by using a gas pressure sensor; controlling an OK light emitting diode indicator to be turned on if a gas pressure value from the gas pressure sensor is equal to or larger than the predetermined value, and controlling an NG light emitting diode indicator to be turned on if the gas pressure value from the gas pressure sensor is not equal to or larger than the predetermined value; and indicating a determined result that represents whether the leakage test is OK or NG based on the light emitting diode indicators as first and second light emitting diode indicators.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An endoscope comprising:
an endoscope body defined by an insertion portion and an operating portion both of which being a hermetically sealed structure with a predetermined gas pressure maintained in the endoscope body;
the operation portion includes a ventilation inlet attached thereto to deliver a gas into the endoscope body and to hold the gas at the predetermined gas pressure in the endoscope body;
a measuring unit configured to be mounted on a board disposed in the operating portion for measuring a gas pressure in the endoscope body; and
a controller configured to acquire a value measured by the measuring unit and to determine whether or not there is a gas leakage from the endoscope body based on the acquired measured value.

2. The endoscope of claim 1 further comprising:
an indicator unit indicating a determined result from the controller.

3. The endoscope of claim 2, wherein the indicator unit is defined by respective first and second LED indicators.

4. The endoscope of claim 1, wherein the measuring unit is defined by a gas pressure sensor.

5. An endoscope comprising:
an endoscope body defined by an insertion portion and an operating portion both of which being a hermetically sealed structure with a predetermined gas pressure maintained in the endoscope body;
the insertion portion includes a light source applying illuminating light into a body cavity;
the insertion portion includes an image capturing unit that captures an image of an inside of the body cavity formed by a return light from the light source;
a communication unit transmitting the image captured by the image capturing unit;
a power supply generates electric power required for endoscopic observations;
the operation portion includes a ventilation inlet attached thereto to deliver a gas into the endoscope body and to hold the gas at the predetermined gas pressure in the endoscope body;
a measuring unit configured to be mounted on a board disposed in the operating portion for measuring a gas pressure in the endoscope body;
a controller configured to acquire a value measured by the measuring unit and to determine whether or not there is a gas leakage from the endoscope body based on the acquired measured value; and
an indicator unit indicating a determined result from the controller.

6. The endoscope of claim 5, wherein
the indicator unit includes a first light emitting diode indicator and a second light emitting diode indicator, and
the controller controls the first light emitting diode indicator or the second light emitting diode indicator to be turned on depending on the determined result.

7. The endoscope of claim 6 further comprising:
an inspection button for inspecting whether or not the predetermined gas pressure is maintained in the endoscope body and
wherein the controller acquires the value measured by the measuring unit when the inspection button is pressed.

8. The endoscope of claim 7 further comprising:
a wireless unit
wherein the controller sends the determined result to an external device through the wireless unit via a wireless link.

9. The endoscope of claim 8, wherein
the measuring unit includes a gas pressure sensor, and
the gas pressure sensor is disposed in a free space area in the operating portion out of interference with mechanisms, wires, and boards laid in the operating portion.

10. A method of indicating a result of a leakage test on an endoscope comprising:
Conducting the leakage test during a standby mode;
determining whether or not a gas pressure in an endoscope body is of a predetermined value by using a gas pressure sensor;
controlling an OK light emitting diode indicator to be turned on if a gas pressure value from the gas pressure sensor is equal to or larger than the predetermined value, and controlling an NG light emitting diode indicator to be turned on if the gas pressure value from the gas pressure sensor is not equal to or larger than the predetermined value; and
indicating a determined result that represents whether the leakage test is OK or NG based on the light emitting diode indicators as first and second light emitting diode indicators.
